(19) United States
(12) Patent Application Publication
(10) Pub. No.: US 2024/0028864 A1
(43) Pub. Date: Jan. 25, 2024

(54) TEMPERATURE ACTUATED MECHANICAL COUNTER

(71) Applicant: Titan Medical Inc., Toronto (CA)

(72) Inventor: Brian Paul Cash, Chapel Hill, NC (US)

(21) Appl. No.: 18/347,316

(22) Filed: Jul. 5, 2023

Related U.S. Application Data
(60) Provisional application No. 63/369,144, filed on Jul. 22, 2022.

(51) Int. Cl. G06M 1/08; A61B 34/30; A61B 90/00

(52) U.S. Cl. CPC G06M 1/083; A61B 34/30; A61B 90/08

(57) ABSTRACT

A temperature actuated mechanical counter is provided. The counter includes a top plate or cover, a base, and an index plate interposed between the top plate and the base. The index plate includes a deflectable plate of a bimetallic material configured to deflect when it reaches a first temperature and configured to revert to an undeflected state when it reaches a second temperature. When the deflectable plate deflects, the index plate moves relative to the top plate and the base and rotates about an axis thereof to a new index location.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The present invention is directed to a temperature actuated mechanical count indicator or counter, and more particularly to an autoclave indicator.

### Description of the Related Art

Products (e.g., tools, instruments) can have a recommended temperature limit for their use. For example, surgical tools can be autoclaved to sterilize them for use, which exposes them to high temperatures.

### SUMMARY

There is a need for a counter that can be exposed to high temperatures (e.g., in an autoclave) and identify how many times a tool or instrument has been exposed to said high temperatures.

In accordance with one aspect of the disclosure, a temperature actuated mechanical counter is provided. The counter includes a top plate or cover, a base, and an index plate interposed between the top plate and the base. The index plate includes a deflectable plate of a bimetallic material configured to deflect when it reaches a first temperature and configured to revert to an undeflected state when it reaches a second temperature. When the deflectable plate deflects, the index plate moves relative to the top plate and the base and rotates about an axis thereof to a new index location.

### BRIEF DESCRIPTION OF THE DRAWINGS

FIG. 1 is a schematic perspective view of a temperature actuated mechanical count indicator or counter.

FIG. 2 is a schematic side view of the temperature actuated mechanical count indicator or counter of FIG. 1.

FIG. 3 is a schematic exploded perspective view of the temperature actuated mechanical count indicator or counter of FIG. 1.

FIG. 4 is another schematic exploded perspective view of the temperature actuated mechanical count indicator or counter of FIG. 1.

FIG. 5 is a schematic exploded side view of the temperature actuated mechanical count indicator or counter of FIG. 1.

FIG. 6 is a schematic cross-sectional view of the temperature actuated mechanical count indicator or counter of FIG. 1.

FIG. 7 is a schematic perspective bottom view of an index plate of the temperature actuated mechanical count indicator or counter of FIG. 1.

FIG. 8 is a schematic view of the temperature actuated mechanical count indicator or counter of FIG. 1 incorporated into a device or tool.

### DETAILED DESCRIPTION

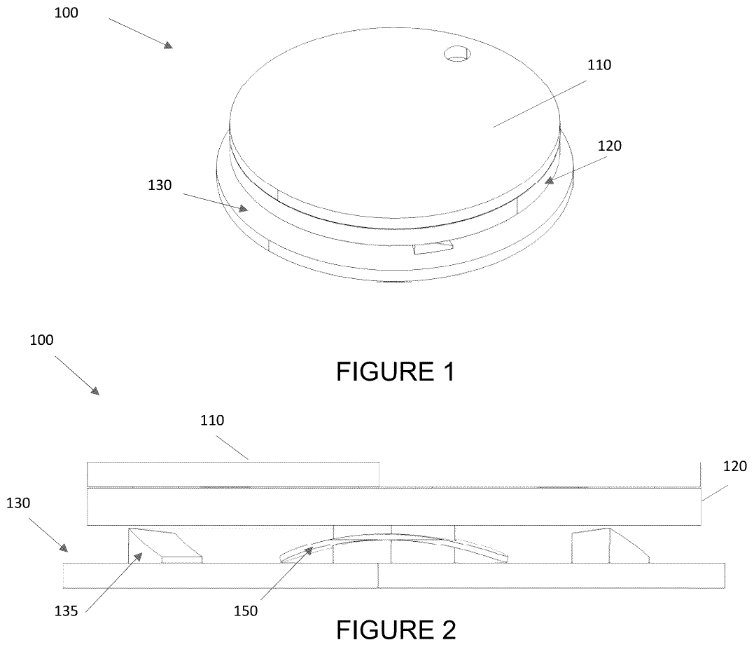

FIGS. 1-6 show a temperature actuated mechanical count indicator or counter 100, which has a top plate or cover 110, a base 130 and an index plate 120 interposed between the top plate 110 and the base 130. As shown in FIG. 6, the top plate 110 can have post 113 (e.g., along a central axis of the top plate 110) that extends from an underside of the top plate 110 and that couples with (e.g., is inserted into) a hollow post 133 of the base 130 (e.g., that extents from an upper side of the base 130) to couple the top plate 110 to the base 130 (e.g., so that the top plate 110 is fixed relative to the base 130). The index plate 120 has an opening 123 (e.g., along a central axis of the index plate 120) that receives the hollow post 133 therethrough, with the post 113 inserted into the hollow post 133, as shown in FIG. 6.

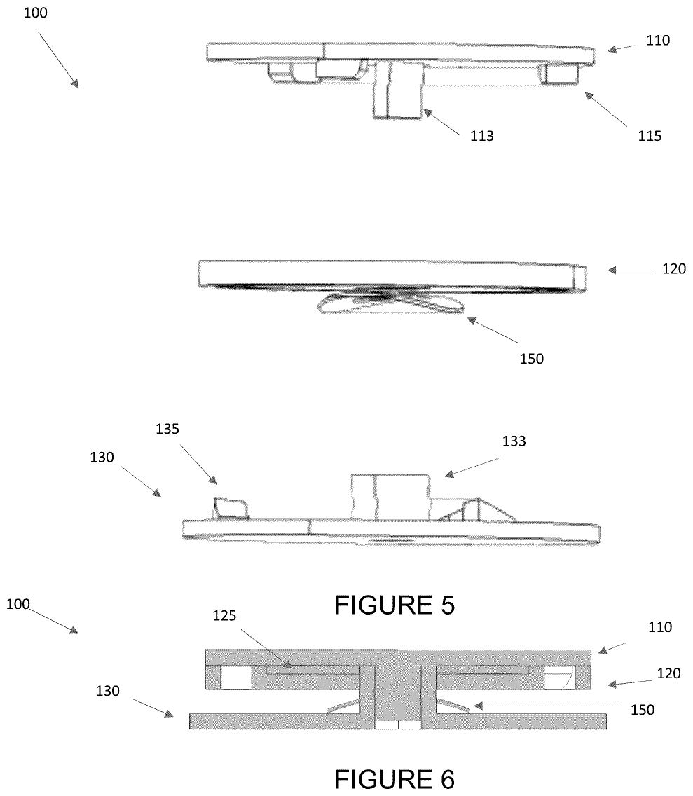

As best shown in FIG. 5, the top plate 110 can have one or more (e.g., multiple, three) index features or teeth 115 that extend from the underside of the top plate 110 and are circumferentially arranged or spaced about the post 113. The base 130 has one or more (e.g., multiple, three) index features or teeth 135 that extend from the upper side of the base 130 and are circumferentially arranged or spaced about the hollow post 133. In one implementation, the teeth 115 can be block shaped (e.g., with a planar horizontal end surface). In on implementation, the teeth 135 can have angled end surfaces. In other implementations, the index features 115, 125 on the top plate 110 and base 130 can have other suitable form factors (e.g., be shaped as bumps).

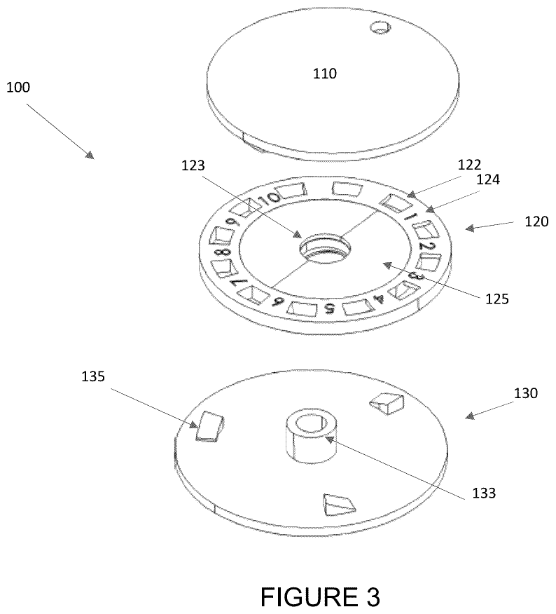

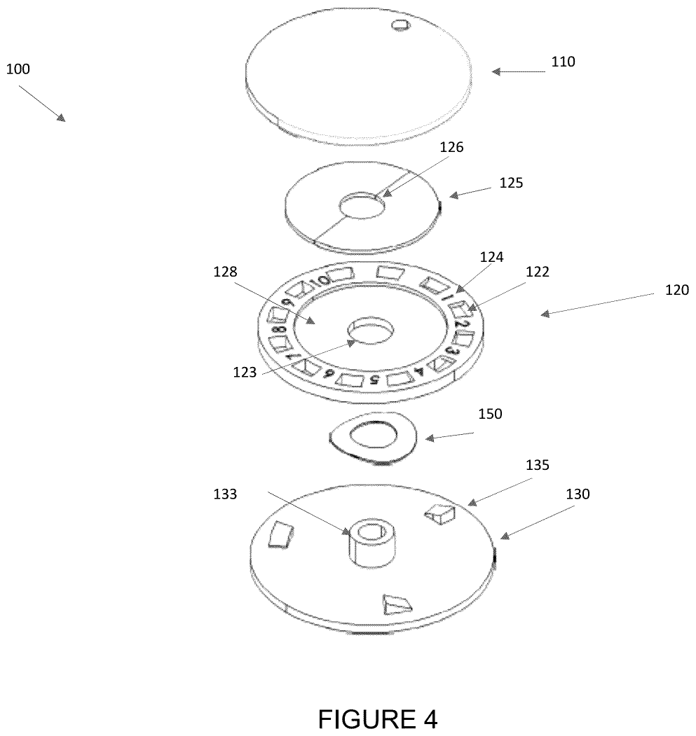

With reference to FIGS. 3-4, a deflectable plate 125 can be disposed on a recessed support 128 of the index plate 120 and has an opening 126 via which the hollow post 133 extends through the deflectable plate 125. The opening 126 aligns with the opening 123 of the index plate 120. In one implementation, the recessed support 128 is excluded and the deflectable plate 125 is retained on the index plate 120 via an interference or coupling of an outer diameter of the deflectable plate 125 and an inner diameter of the index plate 120. In the illustrated implementation, the deflectable plate 125 is disposed between the underside of the top plate 110 and the index plate 120. In the illustrated implementation, a spring 150 (e.g., a Belleville washer) is disposed between an underside of the index plate 120 and an upper side of the base 130 (e.g., disposed about the hollow post 133). In another implementation, the deflectable plate 125 can be disposed between the index plate 120 and the upper side of the base 130 and the spring 150 can be disposed between the underside of the top plate 110 and the index plate 120. In one implementation, the spring 150 can be made of stainless steel. However, the spring 150 can be made of other suitable materials. In one implementation, the top plate 110 and base 130 can be made of a high temperature plastic (e.g., polyetherimide such as ULTEM®, polyetheretherketone, etc.) and can optionally be injection molded.

The deflectable plate 125 can be made of a bimetallic material (e.g., of two or more metals, for example includes different layers of different materials) that deflects (e.g., into a curved, for example convex shape) when it reaches (e.g., is heated to) a first temperature and reverts back to its undeflected state (e.g., a planar or flat shape) when its temperature changes to (e.g., drops below) a second temperature. In one implementation, the second temperature is lower than the first temperature. In another implementation, the second temperature is the same as the first temperature.

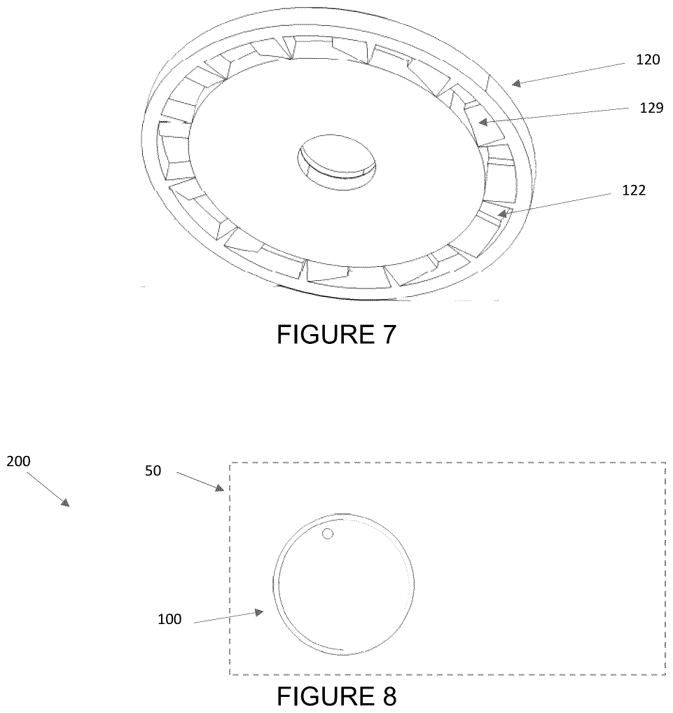

With reference to FIG. 3, the index plate 120 has one or more openings or slots 122 that receive the teeth 115 of the top plate 110, has numerical identifiers 124 along a circumference of the index plate 120. As shown in FIG. 7, the underside of the index plate 120 has angled walls 129 adjacent the slots or openings 122.

In operation, the top plate 110 and base 130 of the counter 100 are fixed relative to each other, and the index plate 120 moves up and down relative to the top plate 110 and base 130 during an indexing operation, as discussed further below. In one example, the deflectable plate 125 deflects when it reaches the first temperature, causing it to bulge and contact and apply a force against the underside of the top plate 110. Such a force causes the index plate 120 to move away from the top plate 110 so that the teeth 115 exit the openings or slots 122 and move toward the base 130, and causes the spring 150 to be compressed between the index plate 120 and the base 130. As the index plate 120 moves toward the base 130, the angled end surface of the teeth 135 of the base engage the angled walls 129 on the underside of the index plate 120, causing the index plate 120 to rotate about the axis of the post 113 and hollow post 133. When the deflectable plate 125 reaches the second temperature and reverts to its undeflected state, the spring 150 exerts a force on the index plate 120 to move the index plate 120 away from the base 130 and toward the top plate 110 so that the teeth 135 extend into different openings or slots 122 different than (e.g., adjacent to) the openings or slots 122 they were before the deflectable plate 125 deflected (e.g., to lock the index plate 120 in a new position or index location relative to the top plate 110.

In this manner, the periodic deflection of the deflectable plate 125 due to exposure of temperature that reach the first temperature, causes the indexing of the index plate 120 between different count locations, allowing the index plate 120 to serve as a counter. In one example, the top plate 110 can have a window or opening via which the current count (e.g., the numerical identifier 124 for the current count) can be viewed. In one example, the indexing (e.g., rotation) of the index plate 120 occurs when the deflectable plate 125 is heated (e.g., to the first temperature), for example to serve as a counter when exposed to heated environments. In another example, the indexing (e.g., rotation) of the index plate 120 occurs when the deflectable plate 125 is cooled (e.g., to the first temperature), for example to serve as a counter when exposed to cooled environments.

In one implementation, the counter 100 can be used to track how many times a tool or instrument is heated (e.g., autoclaved). In one example, the counter 100 can be inserted into the autoclave along with the tool or instrument. In another example, the counter 100 can be attached to the tool or instrument and then autoclaved. In still another example, shown in FIG. 8, the counter 100 can be incorporated into a housing 50 of the tool or instrument 200 (e.g., a surgical tool, a robotic surgical tool). In one example, the counter 100 can be hidden by the housing 50 can only accessible by disassembling the housing 50. In another implementation, the counter 100 can be in a transparent portion of the housing 50 to allow the numerical identifiers 124 to be viewed to indicate the present count. In one implementation, the counter 100 (e.g., the top plate 110, the base 130) can be part of (e.g., be an integral part of, integrated into) the housing 50.

Advantageously, the counter 100 can be used to track how many times a tool or instrument is exposed to temperatures at or above the first temperature (e.g., how many times the tool or instrument is autoclaved). In another example, where the deflectable plate 125 deflects when cooled to or below the first temperature, the counter 100 can be used to track how many times a product is exposed to temperatures at or below the first temperature.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Of course, the foregoing description is that of certain features, aspects and advantages of the present invention, to which various changes and modifications can be made without departing from the spirit and scope of the present invention. Moreover, the devices described herein need not feature all of the objects, advantages, features and aspects discussed above. Thus, for example, those of skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed devices.

What is claimed is:

1. A temperature actuated mechanical counter, comprising:
a top plate having one or more index features on an underside of the top plate and a post extending from the underside of the top plate, the one or more index features circumferentially arranged about the post;
a base having one or more base index features with angled end surfaces on an upper side of the base and a hollow post extending from the upper side of the top plate, the one or more base index features circumferentially arranged about the hollow post, the hollow post configured to couple to the post to fix the top plate relative to the base;
an index plate interposed between the top plate and the base and having numerical identifiers circumferentially arranged about the index plate adjacent corresponding openings or slots in the index plate; and
a deflectable plate attached to the index plate and configured to deflect at a first temperature and to revert to an undeflected state at a second temperature,
wherein deflection of the deflectable plate causes the index plate to move relative to the top plate and the base so that the index plate rotates about an axis of the post to a new index location, and wherein the deflectable plate reverting to the undeflected state causes the index features of the top plate to engage the openings of the index plate to lock the new index location.

2. The counter of claim 1, further comprising a spring between the index plate and the base, the spring configured to apply a force on the index plate when the deflectable plate reverts to the undeflected state to move the index plate.

3. The counter of claim 1, wherein the first temperature is above the second temperature.

4. The counter of claim 1, wherein the first temperature is below the second temperature.

5. The counter of claim 1, wherein the deflectable plate is made of a bimetallic material.

6. The counter of claim 1, wherein the top plate, base and index plate are made of a high temperature plastic material.

7. A robotic surgical tool, comprising:
a housing; and
a temperature actuated mechanical counter housed in the housing, comprising:
a top plate having one or more index features on an underside of the top plate and a post extending from the underside of the top plate, the one or more index features circumferentially arranged about the post;
a base having one or more base index features with angled end surfaces on an upper side of the base and a hollow post extending from the upper side of the top plate, the one or more base index features circumferentially arranged about the hollow post, the hollow post configured to couple to the post to fix the top plate relative to the base;
an index plate interposed between the top plate and the base and having numerical identifiers circumferentially arranged about the index plate adjacent corresponding openings or slots in the index plate; and
a deflectable plate attached to the index plate and configured to deflect at a first temperature and to revert to an undeflected state at a second temperature,
wherein deflection of the deflectable plate causes the index plate to move relative to the top plate and the base so that the index plate rotates about an axis of the post to a new index location, and wherein the deflectable plate reverting to the undeflected state causes the index features of the top plate to engage the openings of the index plate to lock the new index location.

8. The robotic surgical tool of claim 7, further comprising a spring between the index plate and the base, the spring configured to apply a force on the index plate when the deflectable plate reverts to the undeflected state to move the index plate.

9. The robotic surgical tool of claim 7, wherein the first temperature is above the second temperature.

10. The robotic surgical tool of claim 7, wherein the first temperature is below the second temperature.

11. The robotic surgical tool of claim 7, wherein the deflectable plate is made of a bimetallic material.

12. The robotic surgical tool of claim 7, wherein the top plate, base and index plate are made of a high temperature plastic material.

13. The robotic surgical tool of claim 7, wherein the top plate and base are part of the housing.
